(19) United States
(12) Patent Application Publication
(10) Pub. No.: US 2022/0338998 A1
(43) Pub. Date: Oct. 27, 2022

(54) Method for Modeling Glenoid Anatomy and Optimization of Asymmetric Component Design

(71) Applicant: Mayo Foundation for Medical Education and Research, Rochester, MN (US)

(72) Inventor: John W. Sperling, Rochester, MN (US)

(21) Appl. No.: 17/764,027

(22) PCT Filed: Sep. 25, 2020
(86) PCT No.: PCT/US2020/052654

§ 371 Date: Mar. 25, 2022

Related U.S. Application Data
(60) Provisional application No. 62/906,237, filed on Sep. 26, 2019.

(51) Int. Cl. A61F 2/40; A61F 2/30

(52) U.S. Cl. CPC A61F 2/4081; A61F 2/30942; A61F 2002/30784; A61F 2002/30891; A61F 2002/30604

(57) ABSTRACT

Glenoid components with asymmetric fixation points are provided. Also, methods and devices are provided for the optimization of shoulder arthroplasty component design through the use of medical imaging data, such as computed tomography scan data. The methodology may improve the understanding of glenoid anatomy through the use of medical imaging data and 3D modeling, and for glenoid components that exploit this methodology. The methodology provides for how anatomical features change based on the specific location in the glenoid. The methodology can optimize loading and fit at the bone-device interface. Asymmetrical glenoid components are provided with significantly improved initial fixation.

### CROSS-REFERENCE TO RELATED APPLICATIONS

This application claims the benefit of U.S. Provisional Patent Application Ser. No. 62/906,237 filed on Sep. 26, 2019 and entitled “Method for Modeling Glenoid Anatomy and Optimization of Asymmetric Component Design,” which is incorporated herein by reference as if set forth in its entirety for all purposes.

### BACKGROUND OF THE INVENTION

### 1. Field of the Invention

The invention relates to methods for the optimization of joint arthroplasty component design and shoulder arthroplasty components such as glenoid components for joint arthroplasty.

### 2. Description of the Related Art

Various prostheses for the replacement of the shoulder joint are known. In one example shoulder prosthesis, the glenoid is replaced by a glenoid component including a base with a central body as well as protrusions, fins, or pegs that extend from the base into bores formed within the glenoid or holes in the base that allow for screw fixation of the glenoid component to the glenoid. These protrusions or holes can be referred to as fixation sites. When the glenoid component is concave and articulates with a complementary humeral head and stem mounted within the humerus, this type of shoulder prosthesis may be called a “primary”, “total”, or “anatomic” prosthesis. In another example shoulder prosthesis, often called a “reverse” or “inverted” prosthesis, the glenoid component includes a base as well as the attachment of a convex section called a glenosphere that is placed in an opening of the glenoid base that articulates with a complementary concave proximal section of humeral component. In another example shoulder prosthesis, often called a “convertible” glenoid, a glenoid component base is placed and either a concave articular surface for a “total” prosthesis can be placed on the base or a convex glenosphere for reverse arthroplasty can be placed on the base.

There are a breadth of complications in shoulder surgery associated with devices that are not anatomically correct and not optimized for fixation. This includes fracturing the glenoid when trying to implant a device that is not in the shape of the glenoid anatomy, catastrophic early component loosening and risk of infection when contact with native bone is not optimized, as well as lack of long term bone fixation. Thus, there exists a need for improved glenoid components for joint arthroplasty, such as shoulder arthroplasty.

### SUMMARY OF THE INVENTION

The present invention provides a methodology that improves the understanding of glenoid anatomy through the use of CT scan data and 3D modeling, and for glenoid components that exploit this methodology. This methodology describes the interaction of anatomical features of the glenoid and how these features change based on the specific location in the glenoid. Additionally, the methodology has demonstrated that the shape of the glenoid region is side specific. Therefore, having right and left specific devices with an anatomic shape in a true population based distribution further facilitates and improves joint prosthetic component design. The methodology can optimize loading and fit at the bone-device interface. Furthermore, models were created to test the methodology and the interaction of the anatomic features and their interdependence on each other. Asymmetrical glenoid components are provided with significantly improved initial fixation compared to currently available standard symmetrical prosthetic component designs.

Currently, fixation sites for glenoid components are symmetric to allow for one component to be used in both right and left shoulders. In addition, the specific location and length of the fixation sites is not optimized for individual patients. A glenoid component according to the present disclosure with asymmetric fixation points (protrusions or holes for screw fixation) and an optimal shape and size of the central body can maximize stability and preserve bone.

In one aspect, a prosthesis is provided. The prosthesis includes a central body having a longitudinal axis normal to a reference plane that extends through the central body. The prosthesis also includes a plurality of projections extending from an outer surface of the central body. The plurality of projections provide fixation for the prosthesis in a bone of a subject and are asymmetrically spaced in the reference plane around the outer surface of the central body.

In some aspects, a first projection of the plurality of projections has a first projection length, the first projection length defined by a first distance from the central body to an outermost end of the first projection. A second projection of the plurality of projections may have a second projection length, the second projection length defined by a second distance from the central body to an outermost end of the second projection. The second projection length may be less than the first projection length.

In some aspects, the first projection length and the second projection length are asymmetric in a reflection plane along the longitudinal axis. In some aspects, the lengths of each of the plurality of projections are asymmetric in the reflection plane.

In some aspects, the projections are at least one of pegs or screws. At least one of the plurality of projections may have an oblique angle with respect to the reference plane. The plurality of projections may each have an oblique angle with respect to the reference plane and the oblique angles may be asymmetric in a reflection plane along the longitudinal axis.

In some aspects, the central body and the plurality of projections are dimensioned for implantation into a glenoid of a subject. The central body and the plurality of projections may be dimensioned for implantation into a pelvis of a subject. The prosthesis may include four projections, or five projections, six projections, or any number of projections. The prosthesis may be a modular prosthesis.

In another aspect, a prosthesis is provided. The prosthesis includes a central body having a longitudinal axis normal to a reference plane that extends through the central body. The prosthesis also includes a plurality of fixation sites extending through the central body from an outer surface of the central body. The plurality of fixation sites are configured to receive a bone screw to provide fixation for the prosthesis in a bone of a subject. The plurality of fixation sites are asymmetrically spaced in the reference plane around the outer surface of the central body.

In some aspects, a first fixation site of the plurality of fixation sites is configured to receive a first bone screw, which has a first screw length defined by a first distance from the central body to an outermost end of the first screw. A second fixation site of the plurality of fixation sites may be configured to receive a second bone screw, which has a second screw length defined by a second distance from the central body to an outermost end of the second screw. The second screw length may be less than the first screw length. The first screw length and the second screw length may be asymmetric in a reflection plane along the longitudinal axis. The lengths of each bone screw for the plurality of fixation sites may be asymmetric in the reflection plane.

In some aspects, a fixation site may be configured to receive a peg. In some aspects, at least one of the bone screws has an oblique angle with respect to the reference plane. The bone screws may each have an oblique angle with respect to the reference plane and the oblique angles may be asymmetric in a reflection plane along the longitudinal axis.

In some aspects, the central body is dimensioned for implantation into a glenoid of a subject or into a pelvis of a subject. In some aspects, the prosthesis includes four fixation sites, or five fixation sites, or six fixation sites. In some aspects the prosthesis is a modular prosthesis. A glenosphere coupled to the central body may be included for use with a reverse shoulder arthroplasty. A concave articular surface coupled to the central body may be included for use as a convertible glenoid component for shoulder arthroplasty.

In one aspect, a method is provided for manufacturing a prosthetic component for replacing a part of a bone of a joint in a subject. The method includes forming the prosthetic component to include a vertical length and a horizontal length. The vertical length and the horizontal length of the prosthetic component may be determined by creating a three dimensional model from one or more scans of the bone of the joint and positioning on the model a reference plane that extends to an outer surface of the model. The method also includes orienting on an image including the reference plane a first reference line that extends from a first border of the bone to an opposite second border of the bone at a maximum length of a cavity of the bone. The method also includes orienting on the image a second reference line that extends from a third border of the bone to an opposite fourth border of the bone, which is perpendicular to the first reference line in the reference plane at a maximum width of the cavity of the bone. The method also includes orienting on the image a third reference line that extends from a most medial point of the cavity of the bone to the reference plane. The vertical length of the prosthetic component may be determined from a first length of the first reference line. The horizontal length of the prosthetic component may be determined from a second length of the second reference line. A depth of the prosthetic component may be determined from a third length of the third reference line.

In some aspects, the method includes orienting on the image a fourth reference line that extends from the reference plane to a surface of the bone at a location of maximum bone erosion. A thickness of the prosthetic component may be determined from a fourth length of the fourth reference line.

In some aspects, at least one of the plurality of fixation sites is substantially circular. In some aspects, at least one of the plurality of fixation sites has rounded fixation site edges. In some aspects, at least one of the plurality of fixation sites is modular and removeably coupled to the base of the glenoid component. In some aspects, the prosthesis comprises two fixation sites, or three fixation sites, four fixation sites, or any number of fixation sites.

The central body and the plurality of fixation sites can be dimensioned for implantation into a glenoid of a subject. The central body and the plurality of fixation sites can also be dimensioned for implantation into the acetabulum of the hip of a subject.

In some aspects, the prosthesis can be a monoblock prosthesis or a modular prosthesis. The central body and the plurality of fixation sites can be dimensioned for implantation into a glenoid of a subject.

In another aspect, the invention provides a method for manufacturing a prosthetic component for replacing a part of a bone of a joint in a subject. In some aspects, a three dimensional model is created using multiple scans from a single subject or multiple scans from more than one subject. Manufacturing a prosthetic component may include using additive manufacturing. The prosthetic component can be a glenoid component. The prosthesis can be a patient specific implant. In another aspect, the method for manufacturing a prosthetic component may additionally include determining a maximum depth for a prosthetic. The maximum depth and location of the central body as well as the fixation sites can be determined by imaging studies that allow the optimal implant to be placed in a subject.

The prosthetic component can comprise at least one of cobalt chrome, titanium, stainless steel, plastic, and ceramic. The prosthetic component can comprise multiple materials, and one or more additive manufacturing systems may be used to manufacture different parts of the prosthetic component that are assembled for implantation into the patient.

In some aspects, the joint is selected from elbow, wrist, hand, spine, hip, knee, ankle, and foot. When the joint is the elbow, the bone is selected from the ulna, radius and humerus, when the joint is the wrist, the bone is selected from the radius, ulna and carpal bones, when the joint is the hand, the bone is selected from phalanges, metacarpals, and carpals, when the joint is the spine, the bone is a vertebrae, when the joint is the hip, the bone is selected from the femur and the pelvis, when the joint is the knee, the bone is selected from the femur, tibia, and patella, when the joint is the ankle, the bone is selected from the talus, the tibia and the fibula, and when the joint is the foot, the bone is selected from phalanges, tarsals, and metatarsals

These and other features, aspects, and advantages of the present invention will become better understood upon consideration of the following detailed description, drawings, and appended claims.

### BRIEF DESCRIPTION OF THE DRAWINGS

FIG. 1A shows a view of a prior art anatomic glenoid prosthetic component

FIG. 1B shows a view of the prior art reverse glenoid prosthetic component

FIG. 1C is a view of a prior art convertible glenoid component

FIG. 2A is an image of a non-limiting example glenoid without wear.

FIG. 2B is an image of a non-limiting example glenoid with significant wear and bone loss.

FIG. 3A is a front view of a non-limiting example asymmetric left anatomic glenoid component.

FIG. 3B is a front view of a non-limiting example asymmetric right anatomic glenoid component.

FIG. 3C is a perspective view of a non-limiting example asymmetric anatomic glenoid component.

FIG. 3D is a perspective view of a non-limiting example asymmetric anatomic glenoid component with angled projections.

FIG. 3E is a top perspective view of a non-limiting example asymmetric anatomic glenoid component with angled projections.

FIG. 3F is a profile view of a non-limiting example anatomic asymmetric anatomic glenoid component implanted in a bone.

FIG. 4A is a front view of a non-limiting example asymmetric left reverse glenoid component.

FIG. 4B is a front view of a non-limiting example asymmetric right reverse glenoid component.

FIG. 4C is a perspective view of a non-limiting example asymmetric reverse glenoid component.

FIG. 4D is a perspective view of a non-limiting example asymmetric reverse glenoid component with angled fixation screws.

FIG. 4E is a top perspective view of a non-limiting example asymmetric reverse glenoid component with angled fixation screws.

FIG. 5A is a front view of a non-limiting example asymmetric left convertible glenoid component.

FIG. 5B is a front view of a non-limiting example asymmetric right convertible glenoid component.

FIG. 5C is a perspective view of a non-limiting example asymmetric convertible glenoid component.

FIG. 6A is a non-limiting example glenoid shown with non-limiting example reference lines.

FIG. 6B is another non-limiting example glenoid shown with non-limiting example reference lines.

Like reference numerals will be used to refer to like parts from Figure to Figure in the following description of the drawings.

### DETAILED DESCRIPTION OF THE INVENTION

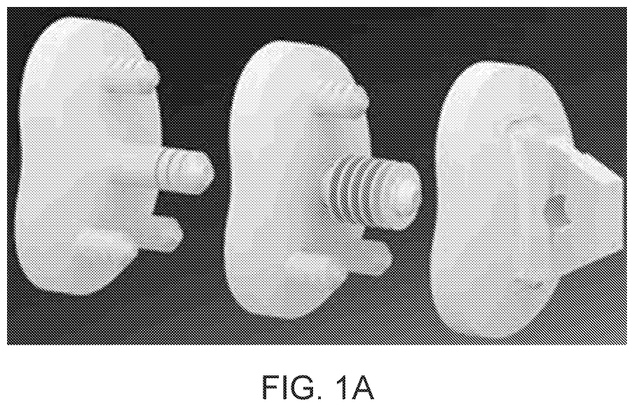

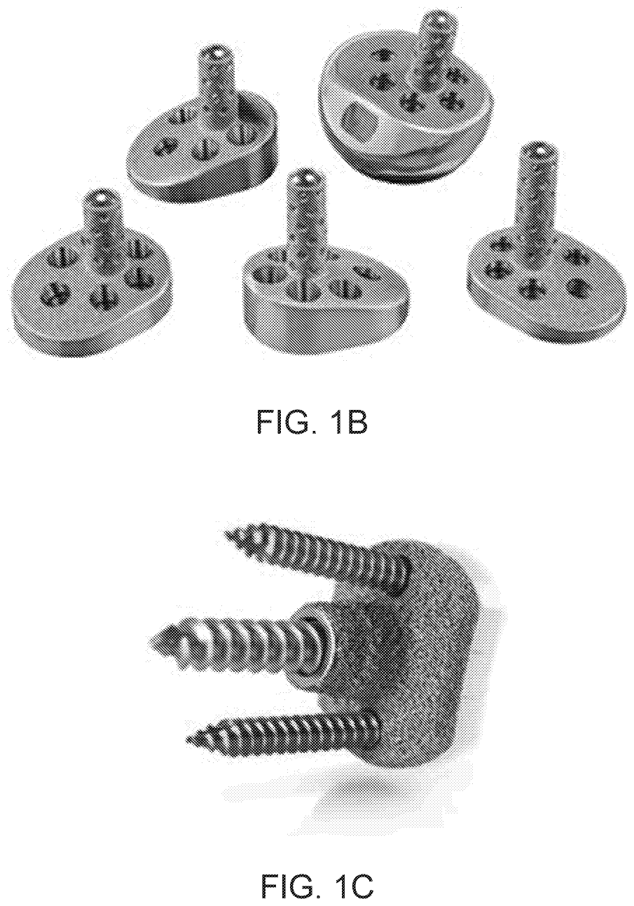

Referring to FIGS. 1A, B, C, a prior art glenoid prosthetic component is shown. FIG. 1A shows a view of a prior art anatomic glenoid prosthetic component. FIG. 1B shows a view of the prior art reverse glenoid prosthetic component. FIG. 1C is a view of a prior art convertible glenoid component. Current prosthetic components are symmetric, pear or rectangular in shape, as can be seen in FIGS. 1A-1C, and not right or left side specific. However, bones are not perfectly pear or rectangular in shape, particularly after there has been wear, which can result in complications and failures when a symmetric component is implanted in such anatomy. Thus, an improved prosthetic component is needed.

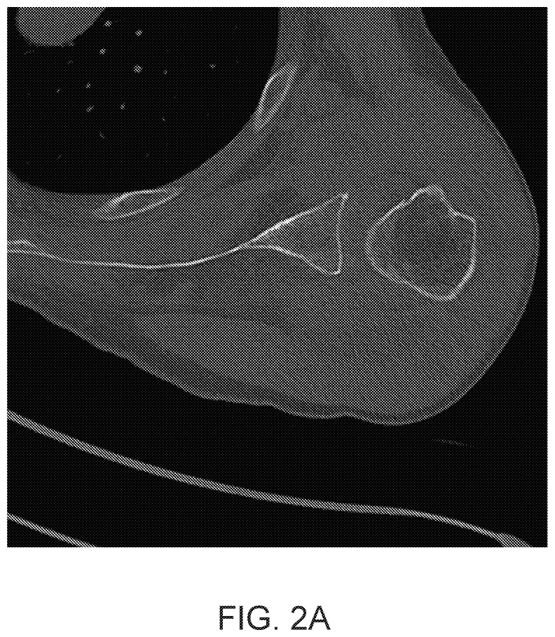

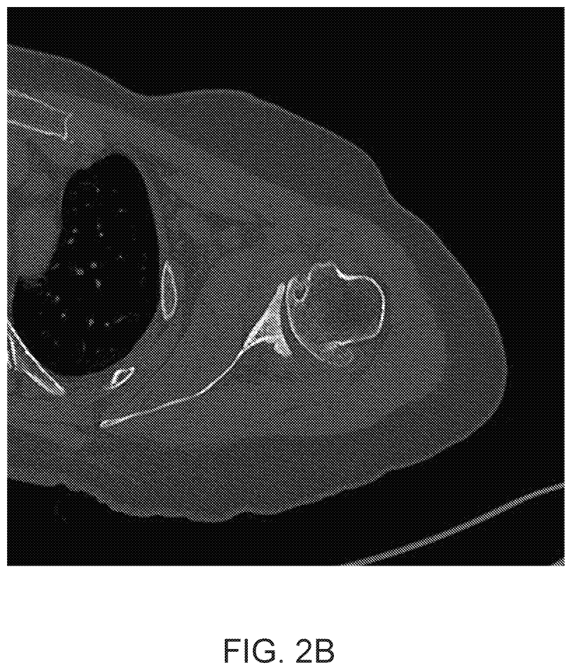

Referring to FIG. 2A, a non-limiting example CT scan of a shoulder, including a glenoid, without wear is shown. Referring to FIG. 2B, a non-limiting example CT scan of a shoulder, including a glenoid, with wear is shown. Currently, there are limited conventional options to manage bone loss, such as the bone loss shown in FIG. 2B. In one non-limiting example, the surgeon may use an augmented component. However, the use of an augmented component necessitates removing additional bone for placement, with limited augment sizes available. An option for managing bone loss in accordance with the present disclosure would be to use a custom glenoid component with asymmetric fixation sites that can preserve bone and maximize fixation.

An asymmetric prosthetic component may include any number of fixation sites. Fixation sites may be selected to provide for optimized fixation between the native bone and the prosthesis, and/or to optimize loading and fit at the bone-device interface. Fixation sites may also be selected to take advantage of higher cancellous or cortical bone quality, or to otherwise prevent prosthesis failure or loosening. Fixation sites may also be selected based upon anatomic considerations, such as the shape of a glenoid vault, to avoid failure at implantation of a prosthesis, such as by breaching a cortical bone perimeter. The fixation sites may be asymmetrically spaced around a central body of the prosthetic component, as described below. The spacing of the fixation sites around the central body may vary depending upon the desired position of the prosthetic component within a patient. The spacing of the fixation sites around the central body may be changed in advance of a procedure when using fixed position fixation sites. Alternately, the use of modular fixation sites may allow for spacing and sizing flexibility during a procedure. The fixation sites may vary in length and size. The length and size of the central body may vary.

In one non-limiting example, the plurality of fixation sites may be described for an asymmetric prosthesis as including at least a first fixation site and a second fixation site. The first fixation site may take the form of a projection from the central body, and may define a first projection length. The first projection length may be defined by a first distance from the center of the central body to an outermost edge of the first projection. Similarly, the second fixation site may take the form of a projection from the central body, and may define a second projection length. The second projection length may be defined by a second distance from the central body to an outermost edge of the second projection. In some non-limiting embodiments, the second projection length may be less than the first projection length.

The prosthetic component may be configured and dimensioned for implantation into a glenoid of a subject. Alternately, the prosthetic component may be configured and dimensioned for implantation into an acetabulum of a subject. The prosthetic component may include any number of fixation sites, such as two fixation sites, three fixation sites, four fixation sites, five fixation sites, or six fixation sites.

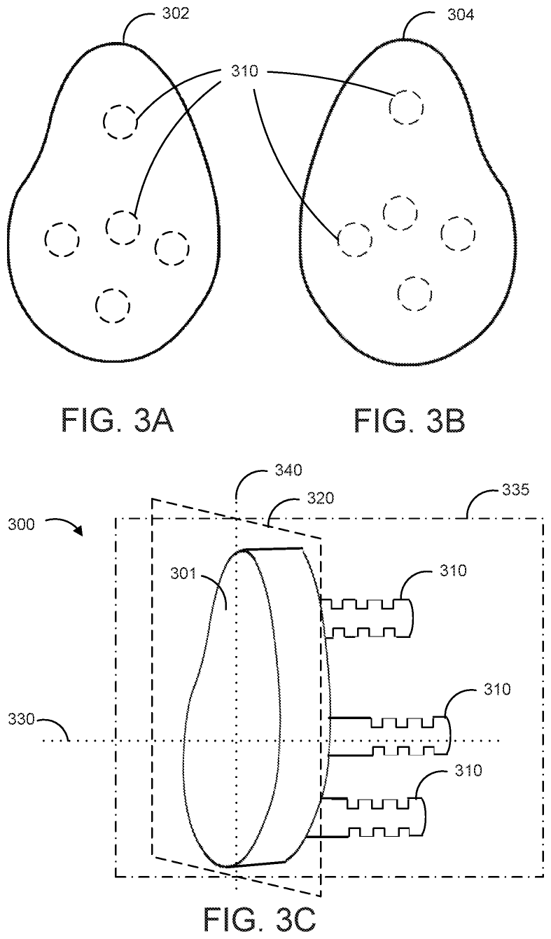

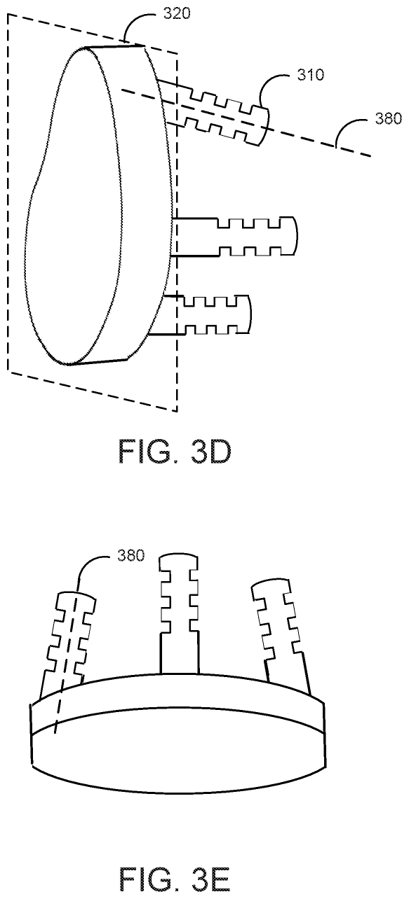

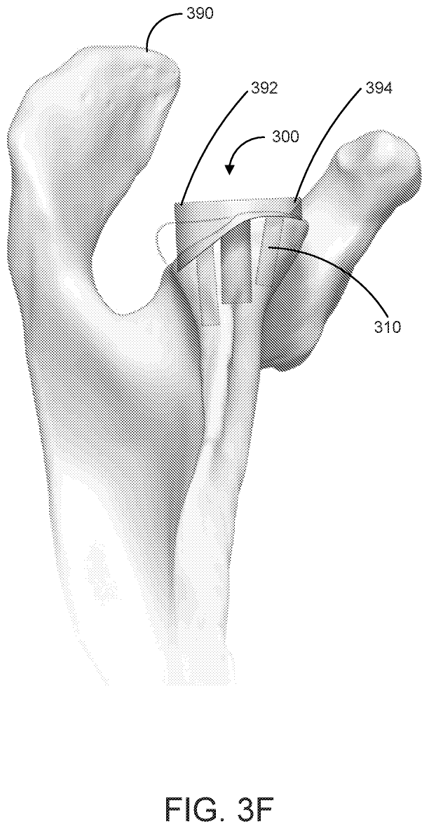

Referring to FIG. 3A-3E, a non-limiting example of an asymmetric anatomic glenoid prosthetic 300 is shown according to the present disclosure. An asymmetric anatomic glenoid prosthetic 300 may include a concave surface 301 that articulates with a complementary humeral head and stem (not shown) mounted within the humerus. This type of shoulder prosthesis may also be called a primary, total, or anatomic prosthesis.

Referring to FIG. 3A, a front view is provided of a left anatomic glenoid, including left anatomic glenoid perimeter 302. Left anatomic glenoid perimeter 302 may form a perimeter of a glenoid prosthetic component 300 that is specific for the left side of a subject. Left anatomic glenoid perimeter 302 may be created to follow a contour or a perimeter of a left glenoid of a subject. Referring to FIG. 3B, a right anatomic glenoid perimeter 304 is shown, which may form a perimeter of a glenoid prosthetic component 300 that is specific for the right side of a subject. The left anatomic glenoid perimeter 302 and right anatomic glenoid perimeter 304 indicate the variation between the left and right side of a subject, and the asymmetric configuration of the glenoid.

Referring to FIG. 3C, a perspective view of the asymmetric anatomic glenoid 300 is shown. Fixation sites may include provisions for screws, or other forms of attachment, or may take the form of pegs, fins, pegs with fins, and the like. Non-limiting example pegs 310 are shown in FIGS. 3A-3E.

Spacing of the pegs 310, may be asymmetric in the reference plane 320. Longitudinal axis 330 is normal to the reference plane 320 and may define an axis of orientation for pegs 310. Lateral axis 340 is perpendicular to the longitudinal axis 330 in the reference plane 320 and may define a major axis of anatomic glenoid 300. A non-limiting example plane of reflection 335 is shown where the left side and the right side of the anatomic glenoid 300 is not mirrored across the plane of reflection 335. Asymmetry indicates that no plane of reflection 335 may be drawn in any orientation of the prosthesis where the prosthesis would be mirrored from a left side to a right side across the plane of reflection 335.

Asymmetry may apply to the locations or numbers of fixation sites, or pegs 310 in the reference plane 320. Asymmetry may apply to the contour of the glenoid perimeter 302 and 304 in the reference plane 320. Asymmetry may apply to the angle of the fixation sites, or pegs 310, such as described in FIGS. 3D and 3E in the plane of reflection 335. Asymmetry may apply to the shape or configuration of the fixation sites, such as a combination of pegs 310 with screw holes forming part of the fixation sites. Asymmetry may apply to the different lengths of pegs 310. One skilled in the art will appreciate that other forms of asymmetry are possible with the present disclosure.

Referring to FIG. 3D, a non-limiting example of an angled peg 310 configuration is shown for the anatomic glenoid 300. The angle 380 of a peg 310 may be determined by the angle of the peg 310 with respect to the normal of the reference plane 320. Any angle 380 may be selected by a user, and may be based upon the criteria described above for fixation sites, such as anatomic constraints, bone quality, or to provide for superior pullout strength to prevent failure of the prosthesis, and the like.

Referring to FIG. 3E, a top-down view of the anatomic glenoid 300 is shown. An angle 380 may be compound, such as being an angle that varies in any orientation from the reference plane 320. In some configurations, multiple pegs 310 may be angled, and the angles may be selected individually for each peg 310. Any number of pegs 310 may be positioned any number of angles 380. The length of the pegs 310 may also be varied and the length of the pegs 310 may be varied with the angle 380 used, such as to define unique angles and unique lengths for each peg 310.

Referring to FIG. 3F, a profile view of the anatomic glenoid 300 is shown implanted into a non-example bone 390. Peg 310 can be seen at an angle and shows consideration of the asymmetric fixation site selection as discussed above. Asymmetry may apply to the conformation of the implant to the bone surface. As can be seen in FIG. 3F, left side 392 is thicker than right side 394 to match the surface of the bone. Similarly, the back of the implant may be shaped in an asymmetric manner across the back's 2D surface in order to conform to peaks and valleys on the surface of the bone 390. In some configurations, the asymmetric shape is nonlinear when conforming to the surface of the bone 390.

In some configurations, at least one of the plurality of fixation sites or pegs 310 may be modular and removeably coupled to the anatomic glenoid 300. Modular fixation sites may allow for a variety of size options.

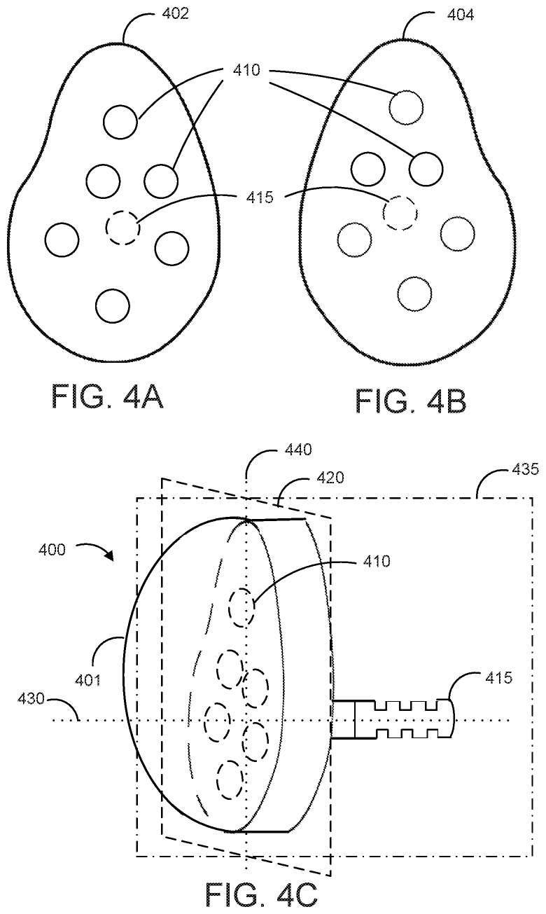

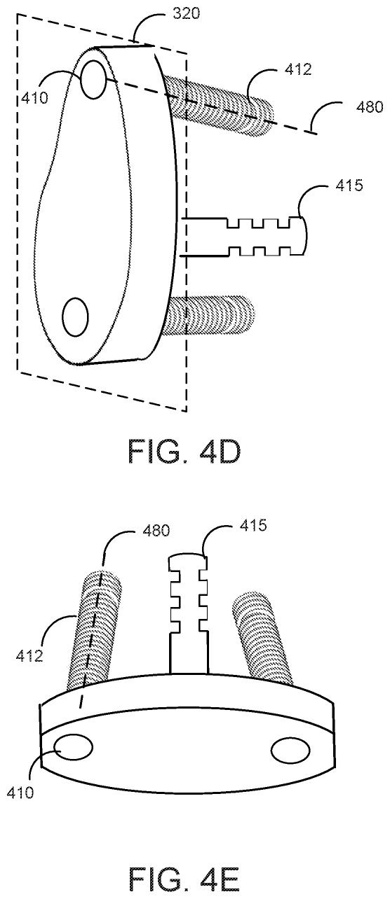

Referring to FIGS. 4A-4E, a non-limiting example of an asymmetric reverse glenoid prosthetic 400 is shown according to the present disclosure. A detailed description of the asymmetric reverse glenoid prosthetic component 400 may be in accordance with the previously described anatomic glenoid prosthetic component 300. An asymmetric reverse glenoid prosthetic 400 may include a convex surface 401 that articulates with a complementary humeral component (not shown) mounted within the humerus.

Referring to FIG. 4A, a front view is provided of a left reverse glenoid, including left reverse glenoid perimeter 402. Left reverse glenoid perimeter 402 may form a perimeter of a reverse glenoid prosthetic component 400 that is specific for the left side of a subject. Left reverse glenoid perimeter 402 may be created to follow a contour or a perimeter of a left glenoid of a subject. Referring to FIG. 4B, a right reverse glenoid perimeter 404 is shown, which may form a perimeter of a glenoid prosthetic component 400 that is specific for the right side of a subject. The left reverse glenoid perimeter 402 and right reverse glenoid perimeter 404 indicate the variation between the left and right side of a subject, and the asymmetric configuration of the glenoid.

Referring to FIG. 4C, a perspective view of the asymmetric reverse glenoid 400 is shown. Fixation sites may include provisions for screws, such as screw holes 410, or other forms of attachment, such as peg 415. Non-limiting example screw holes 410 are shown in FIGS. 4A-4E. Convex surface 401 may be a glenosphere where the glenosphere may include a taper or other attachment, and reverse glenoid 400 may include a provision or hole (not shown) to receive the taper or other attachment mechanism for fixing the glenosphere to the reverse glenoid 400. Asymmetry may apply to the conformation of the implant to the bone surface. In a non-limiting example, a left side may be thicker than a right side to match the surface of the bone. Similarly, the back of the implant may be shaped in an asymmetric manner across the back's 2D surface in order to conform to peaks and valleys on the surface of the bone. In some configurations, the asymmetric shape is nonlinear when conforming to the surface of the bone.

Spacing of the screw holes 410, may be asymmetric in the reference plane 420. As above, asymmetry may apply to reverse glenoid prosthetic 400, such as to the locations or numbers of screw holes 410 in the reference plane 420. Longitudinal axis 430 is normal to the reference plane 420 and may define an axis of orientation for screw holes 410. Lateral axis 440 is perpendicular to the longitudinal axis 430 in the reference plane 420 and may define a major axis of reverse glenoid 400. A non-limiting example plane of reflection 435 is shown where the left side and the right side of the reverse glenoid 400 is not mirrored across the plane of reflection 435. Asymmetry indicates that no plane of reflection 435 may be drawn in any orientation of the prosthesis where the prosthesis would be mirrored from a left side to a right side across the plane of reflection 435. Asymmetry may apply to the angle of the screws 412 that use screw holes 410, or orientation of peg 415, such as described in FIGS. 4D and 4E. One skilled in the art will appreciate that other forms of asymmetry are possible with the present disclosure.

Referring to FIG. 4D, a non-limiting example of an angled screw 412 configuration is shown for the reverse glenoid 400. The angle 480 of a screw 412 may be determined by the angle of the screw 412 with respect to the normal of the reference plane 320. Any angle 480 may be selected by a user, and may be based upon the criteria described above for fixation sites, such as anatomic constraints, bone quality, or to provide for superior pullout strength to prevent failure of the prosthesis, and the like.

Referring to FIG. 4E, a top-down view of the reverse glenoid 400 is shown. An angle 480 may be compound, such as being an angle that varies in any orientation from the reference plane 320. In some configurations, multiple screws 412 may be angled, and the angles may be selected individually for each screw 412. Any number of screws 412 may be positioned at any number of angles 480. Any number of pegs 415 may also be positioned at any number of angles 480. The length of the screws 412 and peg 415 may also be varied and the length of the screws 412 may be varied with the angle 480 used, such as to define unique angles and unique lengths for each screw 412 and/or peg 415.

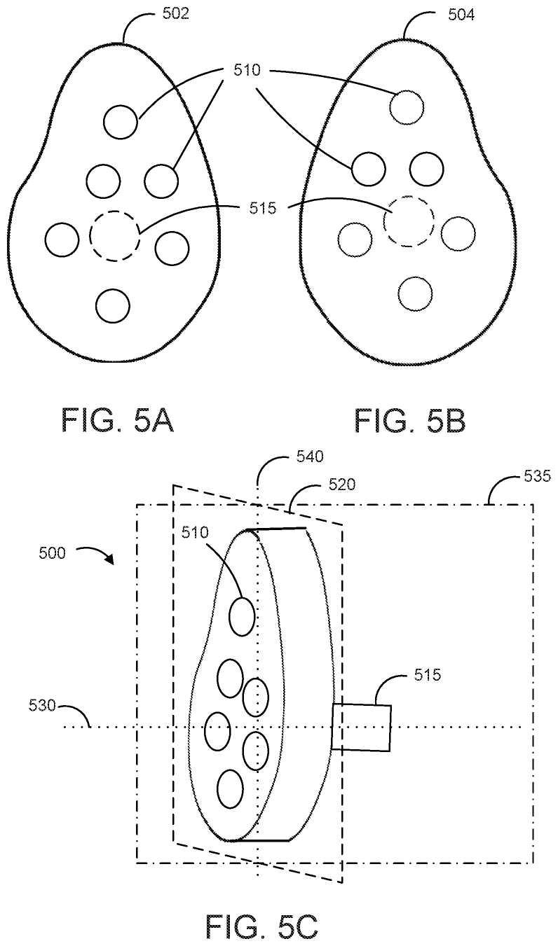

Referring to FIGS. 5A-5C, a non-limiting example of an asymmetric convertible glenoid prosthetic 500 is shown according to the present disclosure. A detailed description of the asymmetric convertible glenoid prosthetic component 500 may be in accordance with the previously described anatomic glenoid prosthetic component 300. A convertible glenoid prosthesis 500 may include either a concave articular surface for a “total” prosthesis or a convex glenosphere for reverse arthroplasty. In some configurations, the asymmetric convertible glenoid component 500 includes a glenosphere where the glenosphere may include a taper or other attachment, and convertible glenoid 500 may include a provision or hole (not shown) to receive the taper or other attachment mechanism for fixing the glenosphere to the convertible glenoid 500. In other configurations, the asymmetric convertible glenoid component 500 includes a concave articular surface where the concave articular surface may include a taper or other attachment, and convertible glenoid 500 may include a provision or hole (not shown) to receive the taper or other attachment mechanism for fixing the concave articular surface to the convertible glenoid 500.

Referring to FIG. 5A, a front view is provided of a left convertible glenoid, including left convertible glenoid perimeter 502. Left convertible glenoid perimeter 502 may form a perimeter of a glenoid prosthetic component 500 that is specific for the left side of a subject. Left convertible glenoid perimeter 502 may be created to follow a contour or a perimeter of a left glenoid of a subject. Referring to FIG. 5B, a right convertible glenoid perimeter 504 is shown, which may form a perimeter of a glenoid prosthetic component 500 that is specific for the right side of a subject. The left convertible glenoid perimeter 502 and right convertible glenoid perimeter 504 indicate the variation between the left and right side of a subject, and the asymmetric configuration of the glenoid.

Referring to FIG. 5C, a perspective view of the asymmetric convertible glenoid 500 is shown. Fixation sites may include provisions for screws, such as screw holes 510, or other forms of attachment, such as pegs 515. Non-limiting example screw holes 510 are shown in FIGS. 5A-5C. Asymmetry may apply to the conformation of the implant to the bone surface. In a non-limiting example, a left side may be thicker than a right side to match the surface of the bone. Similarly, the back of the implant may be shaped in an asymmetric manner across the back's 2D surface in order to conform to peaks and valleys on the surface of the bone. In some configurations, the asymmetric shape is nonlinear when conforming to the surface of the bone.

Spacing of the screw holes 510, may be asymmetric in the reference plane 520. As above, asymmetry may apply to convertible glenoid prosthetic 500, such as to the locations or numbers of screw holes 510 in the reference plane 520. Longitudinal axis 530 is normal to the reference plane 520 and may define an axis of orientation for screw holes 510. Lateral axis 540 is perpendicular to the longitudinal axis 530 in the reference plane 520 and may define a major axis of convertible glenoid 500. A non-limiting example plane of reflection 535 is shown where the left side and the right side of the convertible glenoid 500 is not mirrored across the plane of reflection 535. Asymmetry indicates that no plane of reflection 535 may be drawn in any orientation of the prosthesis where the prosthesis would be mirrored from a left side to a right side across the plane of reflection 535. Asymmetry may apply to the angle of the screws that use screw holes 510, or orientation of pegs 515. One skilled in the art will appreciate that other forms of asymmetry are possible with the present disclosure.

### Example

The following Example has been presented in order to further illustrate the invention and is not intended to limit the invention in any way.

There has been a drive towards bone preserving and less invasive procedures. However, significant deficiencies have been found in currently available devices that do not optimize fixation. Many of the devices used are round with a uniform shape. This results in greater bone removal to make the patients anatomy fit the implant, rather than the implant being designed to match their anatomy. Excessive bone removal and non-optimized fixation sites can result in increased stress at the device bone interface and concern about early catastrophic loosening. This methodology facilitates designing the shape and size of fixation sites for glenoid fixation that are based on the true anatomy of patients undergoing these procedures. Currently, glenoid fixation sites are not anatomic in size, shape, and relative angular position to the glenoid vault.

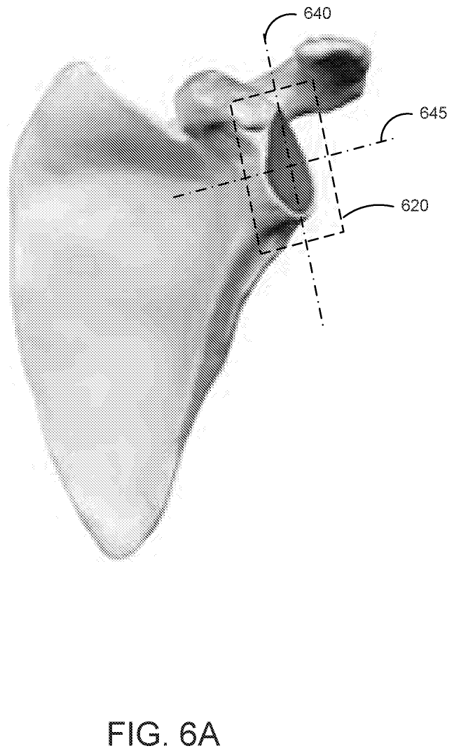

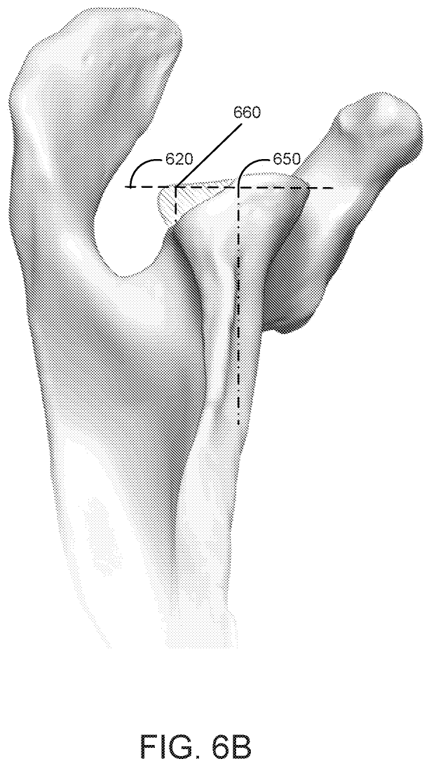

Referring to FIGS. 6A-6B, a non-limiting example glenoid is shown with non-limiting example reference lines. Surface plane 620 may be formed at the surface of a glenoid defined by first reference line 640 and second reference line 645. First reference line 640 may be created by determining a maximum height of a glenoid from a most superior aspect to a most inferior aspect. Second reference line 645 may be created by determining a maximum width of a glenoid from a most anterior aspect to a most posterior aspect. Second reference line 645 meets first reference line 640 at a perpendicular angle. In some configurations, the surface plane 620 is a neutral face plate plane, which compensates for bone erosion or wear by reestablishing a normal anatomic surface of a glenoid.

A third reference line 650 may be created by determining a maximum depth of a glenoid vault from the intersection of the first and second reference lines 640 and 645 that form surface plane 620. Alternatively, third reference line 650 may be created by determining the most medial or deepest point of the glenoid vault and drawing a line to the surface plane 620.

A fourth reference line 660 may be created by determining a location of maximum erosion or wear of a glenoid surface from the surface plane 620, and drawing a line from surface plane 620 to the glenoid surface. Fourth reference line 660 may define a thickness of an implant at the location of maximum erosion.

In one configuration, first reference line 640 determines a maximum height of a prosthesis. Second reference line 645 may determine a maximum width of a prosthesis. Third reference line 650 may determine a maximum depth of a peg or a screw that provides fixation for the prosthesis. Fourth reference line 660 may determine a maximum thickness of a prosthesis.

The development of the unique methodology of this Example can optimize the design of a wide spectrum of shoulder devices and offer a complete glenoid component offering. The methodology ensures that the product designs are correct the first time. By allowing for virtual design and validation, the methodology ensures the proper shaping and sizing of devices based on the true anatomy of patients undergoing these procedures. The methodology can improve the accuracy and efficiency of the design process, saving development cost and accelerating time to market. The methodology describes the interaction of anatomical features of the glenoid, describes how these features change based on the specific location in the glenoid, and demonstrates that the shape is side specific. The methodology demonstrates that right and left specific devices with an anatomic shape in a true population based distribution may further facilitate and improve device design. This optimizes loading and fit at the bone-device interface.

### OTHER APPLICATIONS

The example methodology was performed to further define the glenoid shape and size distribution. It is also evident that implants based on the true anatomy and the described methodology would be beneficial in other areas including hip, knee, ankle, elbow, wrist, hand, and spine.

Thus, this methodology provides for the ability to design glenoid components, including anatomic, reverse, and convertible components for joint arthroplasty, such as shoulder arthroplasty, and methods for the optimization of joint arthroplasty component design.

For any of these applications, a patient specific, custom designed prosthesis may be built based upon image data and the methodology described above for the patient in question. In this way, the prosthetic may be created specifically for the patient using additive manufacturing, or a 3D printer capable of creating a prosthetic out of the required materials, such as cobalt chrome, titanium, stainless steel, or other metals, plastics, ceramics, and the like. If multiple materials are needed to build different components of a modular prosthesis, different 3D printers or other manufacturing methods may be used to make different parts that are then assembled for final implantation into the patient. A number of non-limiting examples of manufacturing techniques such as milling, molding, additive manufacturing, and others can be used as manufacturing systems that could be deployed for building the patient specific devices described above.

The methodology enables the ability to design patient specific asymmetric implants, such as stemless, stemmed, fracture devices, or glenoid replacements, where the surgeon has the ability to preoperatively determine the optimal size, shape, and orientation for a feature, such as a fin/wing/protrusion/body/screw hole, of an implant for a specific patient. The implant can be custom made for an individual patent with custom instrumentation to facilitate intraoperative bone preparation and implantation.

The methodology may be further modified to include an automated process of implant component design where such an automated routine performs measurements automatically on a medical image and optimized implant design features that may be patient specific, or may be used to indicate which size device would be optimally suited for a patient. Automated or manual, the methodology (such as by measuring glenoid depth) may determine the optimal implant, either custom or stock implant, to be selected and ultimately implanted in a patient.

Although what has been described in detail here is with reference to certain embodiments, one skilled in the art will appreciate that the present invention can be practiced by someone other than with the described embodiments, which have been presented for purposes of illustration and not of limitation. Therefore, the scope of the appended claims should not be limited to the description of the embodiments contained herein.

1. A prosthesis comprising:
a central body having a longitudinal axis normal to a reference plane that extends through the central body;
a plurality of projections extending from an outer surface of the central body, wherein the plurality of projections provide fixation for the prosthesis in a bone of a subject and are asymmetrically spaced in the reference plane around the outer surface of the central body.

2. The prosthesis of claim 1 wherein:
a first projection of the plurality of projections has a first projection length, the first projection length defined by a first distance from the central body to an outermost end of the first projection,
a second projection of the plurality of projections has a second projection length, the second projection length defined by a second distance from the central body to an outermost end of the second projection,
wherein the second projection length is less than the first projection length.

3. The prosthesis of claim 2, wherein the first projection length and the second projection length are asymmetric in a reflection plane along the longitudinal axis.

4. (canceled)

5. (canceled)

6. The prosthesis of claim 1, wherein at least one of the plurality of projections is a peg or screw, or has an oblique angle with respect to the reference plane.

7. The prosthesis of claim 6, wherein the plurality of projections each have an oblique angle with respect to the reference plane and the oblique angles are asymmetric in a reflection plane along the longitudinal axis

8. The prosthesis of claim 1, wherein the central body and the plurality of projections are at least one of: dimensioned for implantation into a glenoid of a subject, or dimensioned for implantation into a pelvis of a subject.

9. (canceled)

10. The prosthesis of claim 1, wherein the prosthesis comprises at least one of: four projections, or five projections, or six projections, or is a modular prosthesis.

11. (canceled)

12. A prosthesis comprising:
a central body having a longitudinal axis normal to a reference plane that extends through the central body;
a plurality of fixation sites extending through the central body from an outer surface of the central body, wherein the plurality of fixation sites are configured to receive a bone screw to provide fixation for the prosthesis in a bone of a subject, and
wherein the plurality of fixation sites are asymmetrically spaced in the reference plane around the outer surface of the central body.

13. The prosthesis of claim 12 wherein:
a first fixation site of the plurality of fixation sites is configured to receive a first bone screw, wherein the first bone screw has a first screw length, the first screw length defined by a first distance from the central body to an outermost end of the first screw,
a second fixation site of the plurality of fixation sites is configured to receive a second bone screw, wherein the second bone screw has a second screw length, the second screw length defined by a second distance from the central body to an outermost end of the second screw,
wherein the second screw length is less than the first screw length.

14. The prosthesis of claim 13, wherein the first screw length and the second screw length are asymmetric in a reflection plane along the longitudinal axis.

15. (canceled)

16. The prosthesis of claim 12, further comprising a fixation site configured to receive a peg.

17. The prosthesis of claim 12, wherein at least one of the bone screws has an oblique angle with respect to the reference plane.

18. The prosthesis of claim 17, wherein the bone screws each have an oblique angle with respect to the reference plane and the oblique angles are asymmetric in a reflection plane along the longitudinal axis.

19. The prosthesis of claim 12, wherein the central body is at least one of: dimensioned for implantation into a glenoid of a subject, or is dimensioned for implantation into a pelvis of a subject.

20. (canceled)

21. The prosthesis of claim 12, wherein the prosthesis comprises at least one of: four fixation sites, or five fixation sites, or six fixation sites or is a modular prosthesis.

22. (canceled)

23. The prosthesis of claim 12, further comprising at least one of: a glenosphere coupled to the central body for use with a reverse shoulder arthroplasty, or a concave articular surface coupled to the central body for use as a convertible glenoid component for shoulder arthroplasty.

24. (canceled)

25. A method for manufacturing a prosthetic component for replacing a part of a bone of a joint in a subject, the method comprising:
forming the prosthetic component to include a vertical length and a horizontal length, the vertical length and the horizontal length of the prosthetic component having been determined by
(a) creating a three dimensional model from one or more scans of the bone of the joint;
(b) positioning on the model a reference plane that extends to an outer surface of the model;
(c) orienting on an image including the reference plane a first reference line that extends from a first border of the bone to an opposite second border of the bone at a maximum length of a cavity of the bone;
(d) orienting on the image a second reference line that extends from a third border of the bone to an opposite fourth border of the bone, wherein the second reference line is perpendicular to the first reference line in the reference plane at a maximum width of the cavity of the bone;
(e) orienting on the image a third reference line that extends from a most medial point of the cavity of the bone to the reference plane;
(f) determining the vertical length of the prosthetic component from a first length of the first reference line;
(g) determining the horizontal length of the prosthetic component from a second length of the second reference line;
(h) determining a depth of the prosthetic component from a third length of the third reference line.

26. The method of claim 25, wherein the cavity of the bone is the glenoid cavity.

27. The method of claim 25, further comprising orienting on the image a fourth reference line that extends from the reference plane to a surface of the bone at a location of maximum bone erosion.

28. The method of claim 27, further comprising determining a thickness of the prosthetic component from a fourth length of the fourth reference line.
